(19) United States
(12) Patent Application Publication
(10) Pub. No.: US 2009/0240158 A1
(43) Pub. Date: Sep. 24, 2009

(54) User-interface for displaying fetal condition information during labor and apparatus implementing same

(75) Inventors: Emily Hamilton, Verdun (CA); Maciej Macieszczak, Kanata (CA)

Correspondence Address: DOWELL & DOWELL P.C., 103 Oronoco St., Suite 220, Alexandria, VA, 22314, US

(73) Assignee: LMS Medical Systems Ltd, Montreal (CA)

(21) Appl. No.: 12/285,617

(22) Filed: Oct. 9, 2008

Related U.S. Application Data
(60) Provisional application No. 60/996,935, filed on Dec. 11, 2007.

(51) Int. Cl. A61B 5/0444

(52) U.S. Cl. 600/511

(57) ABSTRACT

A computer readable storage medium storing a program element for execution by a computer to implement a user interface. The user interface conveys fetal condition information to a user. The user interface has a first section showing one or more vital signs of a fetus. The first section includes at least one tracing associated with a vital sign of the fetus and a marker to visually highlight an area of the tracing corresponding to an event of interest. The first section also has a control component that can be activated by the user to deliver additional information about the event of interest.

### CROSS-REFERENCE TO RELATED APPLICATION

This application claims the benefit under 35 USC §120 of U.S. provisional patent application Ser. No. 60/996,935 filed Dec. 11, 2007 by E. Hamilton and which is presently pending. The contents of the above-mentioned patent application are incorporated herein by reference.

### FIELD OF THE INVENTION

The invention relates to information delivery and more particularly to a user interface to show FHR variations and contraction information during labor.

### BACKGROUND

During labor, uterine contractions push the baby through the birth canal. Uterine contractions can also reduce oxygen delivery to the baby by causing compression of the umbilical cord or by reducing maternal blood flow through the uterus and placenta. These intermittent stresses are usually mild and well tolerated by the baby. However, under certain conditions the baby will be subject to excessive oxygen deprivation. If this is sufficiently prolonged or severe it can lead to fetal brain injury or death.

Fetal Heart Rate (FHR) and maternal contraction monitoring is performed in approximately 90% of labors. The pattern of the FHR response to contractions reflects in part the in-utero mechanism producing the FHR change as well as the ability of the fetus to respond and compensate. Other aspects of the FHR recording, such as the heart beat to heart beat variation in the FHR reflect the ability of the central nervous system to modulate the fetal heart. Professional bodies have defined the size and shape characteristics of several deceleration and acceleration patterns.

Clinicians visually review these monitor recordings, including the trends in patterns over time, and integrate this with other pertinent clinical information to assess fetal tolerance to labor and risk of hypoxic injury. Although deceleration patterns are defined, numerous studies show inconsistent labeling by clinicians especially when the patterns are mixed and tracings are neither completely normal nor highly abnormal. Visual inspection of the tracing is an imprecise way to measure these fluctuations in the FHR. Integrating information consistently over many hours is a challenging task for humans and well known to be further exacerbated by fatigue, distraction and inexperience. Several reviews of hypoxic fetal death or brain injury confirm a high rate of medical error ranging from 40 to 60%, centering most often on failure to appreciate the degree and duration of abnormality.

Therefore, a need exists in the industry to provide improved FHR information delivery mechanisms allowing the physician during labor to identify at a glance periods of FHR changes, without the need to browse through lengthy recordings in an attempt to spot trends.

### SUMMARY

As embodied and broadly described herein the invention provides a computer readable storage medium holding a program element for execution by a computer to implement a user interface conveying fetal condition information to a user, the user interface comprising a section showing one or more vital signs of a fetus, said first section including:
a) at least one tracing associated with a vital sign;
b) a marker to visually highlight an area of the tracing corresponding to an event of interest;
c) a control component that can be activated by the user to deliver additional information about the event of interest.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

A detailed description of examples of implementation of the present invention is provided hereinbelow with reference to the following drawings, in which:

FIG. 1 shows a high-level functional block diagram of a system including an apparatus 100 for monitoring an obstetrics patient in accordance with a specific example of implementation of the present invention;

FIG. 2 shows a block diagram of the apparatus 100 depicted in FIG. 1 in accordance with a specific example of implementation of the present invention;

FIG. 3 show a specific example of implementation of a graphical user interface implemented by the system shown in FIG. 1 for providing fetal condition information in accordance with a first non-limiting example of implementation of the invention;

FIG. 4 is a block diagram of an apparatus for providing fetal condition information in accordance with a specific example of implementation of the present invention;

FIG. 5 is a high level conceptual block diagram of a program element for implementing a graphical user interface of the type shown in FIG. 3 in accordance with a specific example of implementation of the present invention;

FIG. 6 shows a functional block diagram of a client-server system for providing fetal condition information in accordance with an alternative specific non-limiting example of implementation of the present invention.

In the drawings, embodiments of the invention are illustrated by way of example. It is to be expressly understood that the description and drawings are only for purposes of illustration and as an aid to understanding, and are not intended to be a definition of the limits of the invention.

### DETAILED DESCRIPTION

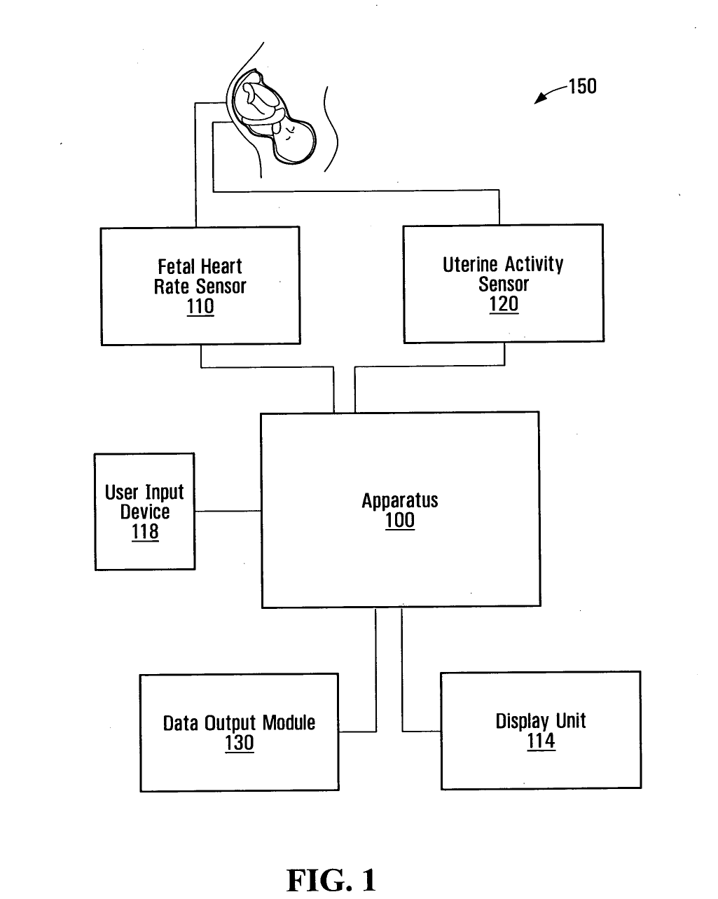

With reference to FIG. 1, there is shown a configuration of a system 150 for monitoring labor progress for an obstetrics patient comprising a fetal heart rate sensor 110, a uterine activity sensor 120, a user input device 118, an apparatus 100 implementing a user interface for displaying labor related information and a display unit 114.

The user input device 118 is for receiving data from a user of the system. The user input device 118 may be used, for example, to enter information associated with the obstetrics patient and/or to manipulate the information displayed by the user interface implemented by the apparatus 100. The type of data received through input device 118 may vary depending on the type of information that the apparatus 100 is adapted to process and interpret. Specific examples of the type of information that may be provided through input device 118 will be described later on in the specification. The user input device 118 includes any one or a combination of the following: keyboard, pointing device, touch sensitive surface, actuator/selection switches or speech recognition unit.

The fetal heart rate sensor 110 is for detecting a fetal heart rate of a fetus in-utero, also referred to as a fetus in the womb. The fetal heart rate sensor 110 samples the fetal heart rate at a certain pre-determined frequency to generate the signal indicative of the fetal heart rate. Fetal heart rate sensors are well known in the art to which this invention pertains and any suitable sensor for detecting a fetal heart rate may be used without detracting from the spirit of the invention and as such will not be described further here.

The uterine activity sensor 120 is for monitoring uterine activity (TOCO). The sensor samples the contraction pattern at a certain pre-determined frequency to generate the signal indicative of uterine activity. Sensors for monitoring uterine activity are well known in the art to which this invention pertains and any suitable sensor may be used without detracting from the spirit of the invention and as such will not be described further here.

Optionally, the monitoring system 150 may include other sensors (not shown) for measuring labor progress and the fetus' tolerance to labor. Such sensors may include for example:
a sensor for measuring the maternal oxygen saturation
a sensor for measuring the fetal oxygen saturation
a sensor for measuring maternal blood pressure

Suitable sensors other than the ones described above may also be used without detracting from the spirit of the invention.

The display unit 114 is coupled to the apparatus 100 and receives a signal causing the display unit 114 to display a graphical user interface module implemented by apparatus 100. The display unit 114 may be in the form of a display screen, a printer or any other suitable device for conveying to the physician or other health care professional the progression of labor related information. In a non-limiting implementation, the display unit 114 includes one or more display screens to display the graphical user interface. The display unit 114 may also include a printer device for providing a paper print out of the graphical user interface implemented by apparatus 100. In embodiments where the display unit 114 is in the form of a display screen, it may be part of any suitable type of apparatus including, without being limited to, a desktop/laptop computing apparatus, a personal digital assistant (PDA), a telephone equipped to video display capability, a TV monitor or any other suitable device equipped with a display screen for visually conveying information to a user.

Optionally, the monitoring system 150 may further include a data output module 130. The data output module 130 is in communication with the apparatus 100 and is suitable for receiving signals generated by the apparatus 100. In a first specific example of implementation, the data output module 130 includes an audio module for releasing audio signals on the basis of signals received from the apparatus 100. In a second specific example of implementation, the data output module 130 includes a data communication entity suitable for transmitting messages to remote devices causing the latter to convey to a user of the monitoring system 150 fetal condition information and, optionally, maternal condition information. Examples of remote devices include, without being limited to, PDAs, telephones, pagers and computing terminals.

### Apparatus 100

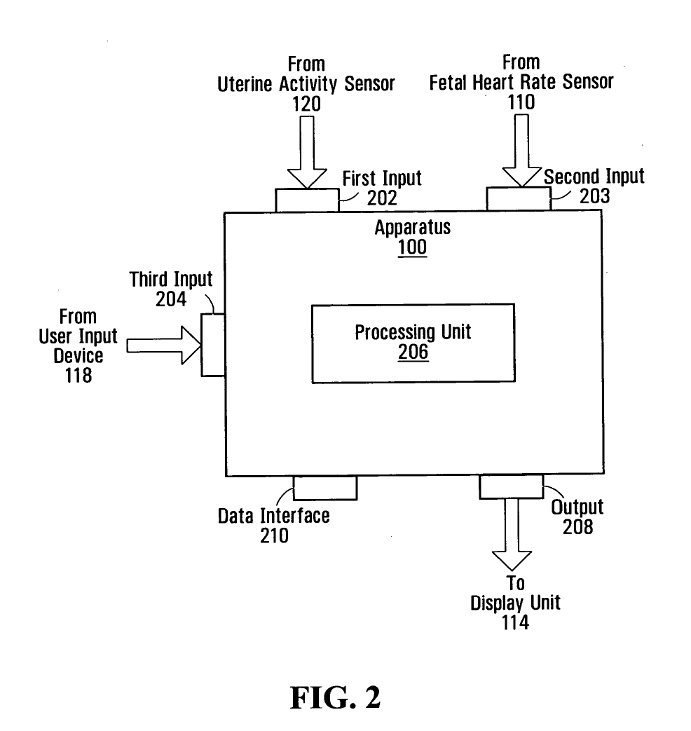

A specific example of implementation of apparatus 100 will now be described with reference to FIG. 2. The apparatus 100 includes a first input 202, a second input 203, a processing unit 206 and an output 208. The first input 202 is for receiving a contraction signal originating from the uterine activity sensor 120 (shown in FIG. 1) and conveying information related to occurrences of uterine contractions over time. The second input 203 is for receiving a fetal heart rate signal as generated by fetal heart rate sensor (110). It will be readily appreciated that, although the fetal heart rate signal and the uterine activity signal are received at different inputs in the embodiment illustrated in FIG. 2, these signals may be provided to processing unit 206 through a same input without detracting from the spirit of the invention. Optionally, as shown in FIG. 2, the apparatus further includes a third input 204 for receiving data from a user through input device 118 (shown in FIG. 1).

The processing unit 206 processes the signals received at inputs 202 and 203 as well as the user data received at input 204 and to derive various information elements including fetal condition information and, optionally, maternal condition information. The processing unit 206 also implements a graphical user interface module for displaying the device information elements. The output 208 is for releasing a signal for causing display unit 114 to display the graphical user interface module implemented by processing unit 206.

The output 208 is for releasing a signal for causing the display unit 114 (shown in FIG. 1) to display the graphical user interface implemented by processing unit 206. Optionally still, the apparatus further includes a data interface 210 for exchanging signals with the data output module 130 (shown in FIG. 1) for causing the latter to convey fetal condition information and, optionally, maternal condition information to a user of the labor monitoring system 150 (shown in FIG. 1).

### Processing Unit 206

The processing unit 206 receives data from the fetal heart rate sensor 110, the uterine activity sensor 120 and the input device 118 and processes that data to derive therefrom various information elements including fetal condition information and, optionally, maternal condition information.

In a specific example of implementation, the processing unit 206 includes functionality for deriving fetal heart rate feature measurements. Fetal heart rate feature measurements include, for example, mean baseline, mean baseline variability, decelerations and accelerations amongst others. Any suitable algorithm for identifying fetal heart rate feature measurements may be used. Any suitable pattern recognition technique may be used for identifying the occurrence of acceleration/deceleration events and for calculating the baseline for the fetal heart rate. Such techniques are well known in the art of signal processing and as such will not be described further here. The processing unit 206 also includes functionality for processing the contraction signal received at second input 203 to detect the occurrence of contraction events. Any suitable pattern recognition technique may be used for identifying the occurrence of contraction events. Such techniques are well known in the art of signal processing and as such will not be described further here.

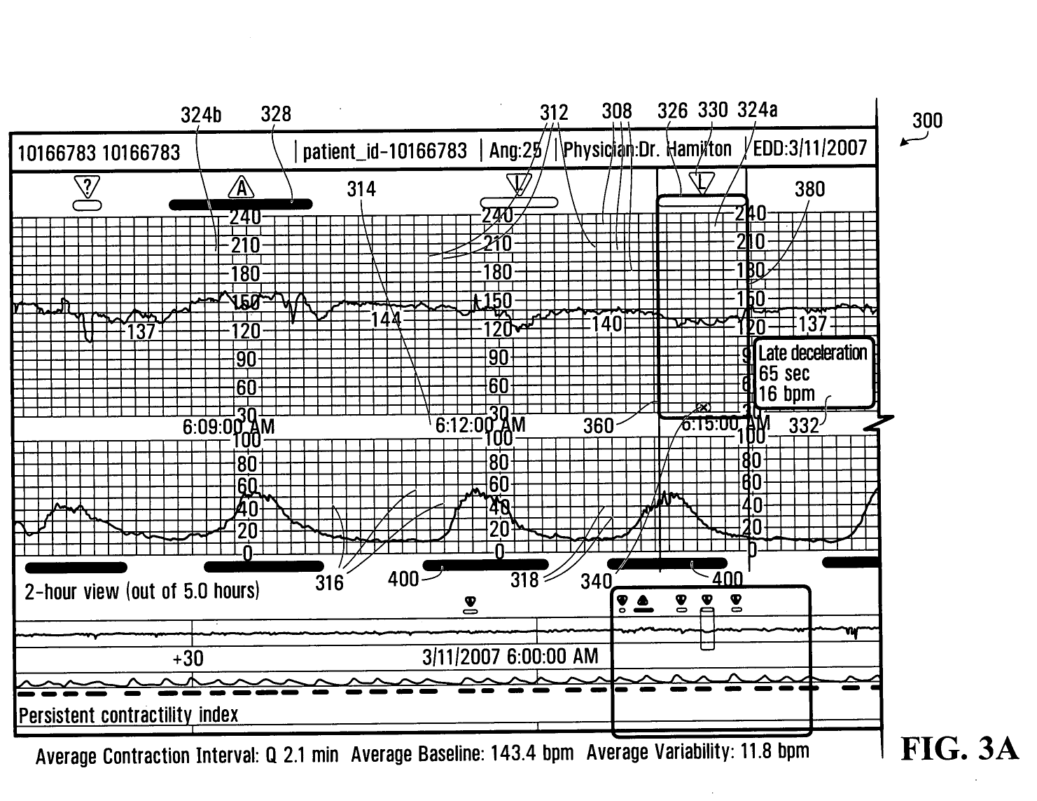

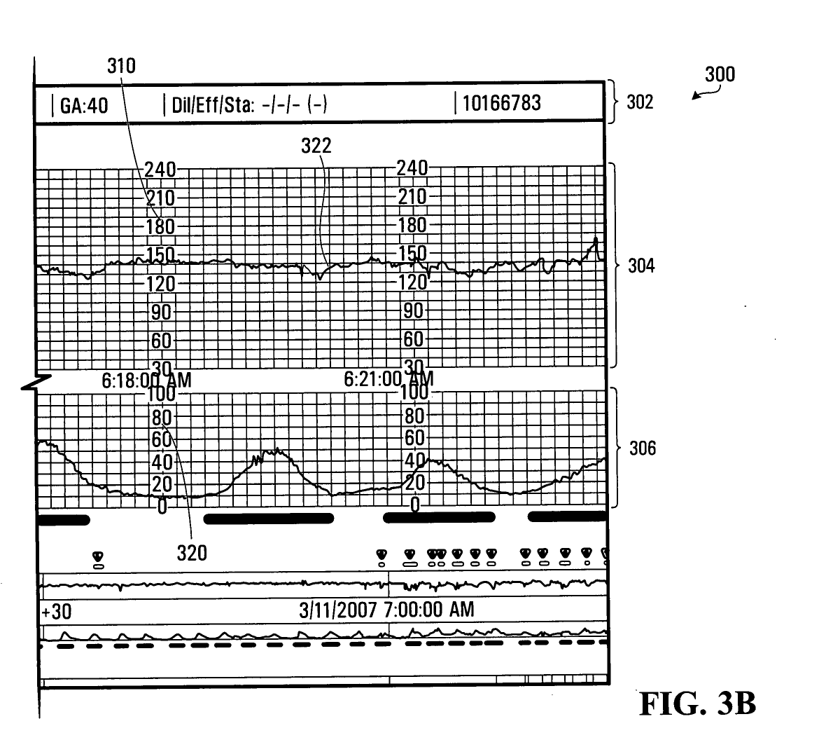

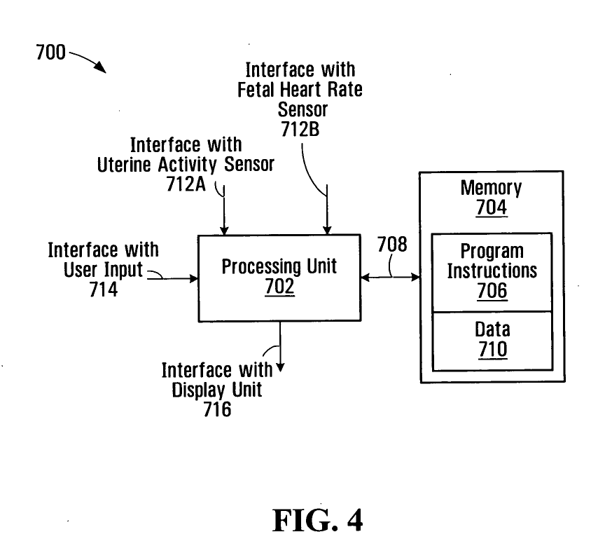

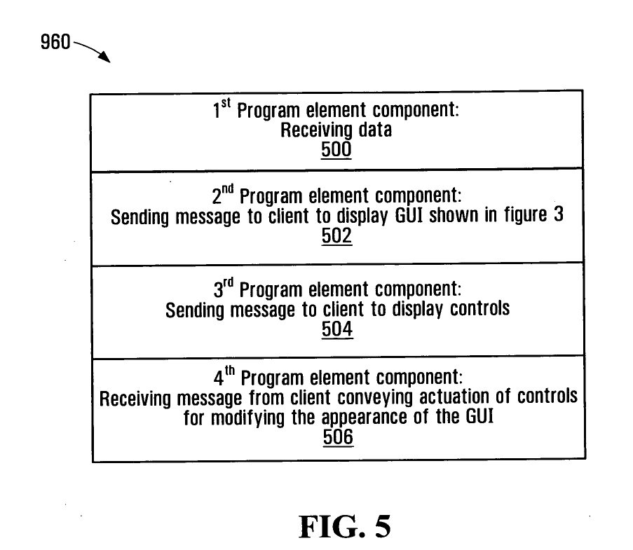

FIG. 3 shows an example of a user interface, as it would appear on the display unit 114 while an obstetrics patient is in labor. The user interface 300 delivers visual information to the user and, at the same time, accepts user inputs via the user input device 118. The inputs via user input device help further refine the usefulness and accuracy of the information displayed on the display unit 114.

In the non-limiting example depicted, an upper section 304 of the user interface 300 is used primarily to display fetal heart rate information and, optionally, maternal heart rate information. A lower section 306 of the user interface is used primarily to display maternal uterine contraction information and, optionally maternal and/or fetal oxygen saturation information.

Specifically, the user interface 300 has a top information bar 302 that contains basic information about the ongoing procedure, in particular the date of the procedure, the identification of the patient and the physician identification among others. Below the information bar 302, the display is divided into two separate sections 304 and 306. The sections 304 and 306 are synchronized with respect to time; in other words they share a common time line. Accordingly, an event in the upper section 304 that is aligned with an event in the lower section 306 occurs at the same point in time.

The drawings show sections 304, 306 aligned horizontally. This is a useful disposition of the information since it mimics the way a chart is laid on a sheet and is well suited for computer monitors set in a landscape orientation. However the information can also be presented differently, especially on devices that are not intended to be used in a landscape orientation, such as portable devices that have screens oriented portrait wise. In this instance, the sections 304, 306 can run vertically, instead of running horizontally.

Also note that while the time synchronization between the sections 304 and 306 is considered to be an advantageous feature, it is not absolutely required and may be dispersed without departing from the spirit of the invention. Specifically, the each one of the sections 304, 306 can be shown on two different display units 114, which may be located side by side.

The upper section 304 is provided with two sets of grid lines. The horizontal set of gridlines 308 provide reference to a heart beat axis 310, which in the example shown ranges from 30 beats per minute to 240 beats per minute. The vertical set of gridlines 312 is associated with a time scale 314 which is shown at the bottom of the upper section 304. The time scale 314 appears as a bar showing the actual time of day.

Below the upper section 304, which is used primarily to display fetal heart rate information, appears the lower section 306. The lower section 306, which is used primarily to convey maternal uterine contraction information, is similarly arranged to the upper section 304 in that it has vertical grid lines 316 that refer to the time scale 314, which is common to both sections 304 and 306.

Horizontal grid lines 318 are also provided but they refer to the contractions strength axis 320. In the example shown the contractions strength axis 320 ranges from 0 to 100.

The upper section includes a tracing 322 showing the variation of the fetal heart beat with respect to time. While this tracing provides very useful information to the physician, it may be difficult to interpret sometimes as the minute variations in the tracing make longer terms trends less obvious to see. As an assist to FHR tracing 322 interpretation the upper section 304 includes markers that visually indicate the occurrence of FHR significant events. One such event is an FHR downward or upward change of significant size and duration.

In the example shown, the markers are in the form of windows (several such windows are shown in the drawing denoting several events of interest). For the purpose of the example consider the window 324a. That window is laid over the tracing 322 and is rendered in a color that is different from the background of the chart such to make it stand out visually. The coloring of the window 324a is translucent. In this fashion the tracing 322 remains visible within the frame of the window 324a. The horizontal size of the window denotes the duration of the event of interest, in this case a reduction of the FHR. Indeed, it can be clearly noted that the tracing 322 dips below the 140 beats per minute line.

The window 324a shows a deceleration of the FHR. Windows that show acceleration, such as the window 324b appear in a different color. In the example shown the window 324b is in green, which by convention is normally associated to a “safe” event, while the amber color used in the case of the window 324a denotes an “unsafe” event. The reader will appreciate that the particular color chosen for the window is a matter of design and many different color schemes can be used without departing from the spirit of the invention.

In addition to using color to highlight the windows 324a, 324b, other techniques can also be employed such as causing the window to flash to further attract the physician's attention. Audible alarms can be also be employed in extreme circumstances.

To reduce clutter on the display, windows, such as windows 324a and 324b, can initially appear in the form of bars 326 and 328 having a color that denotes the nature of the event to the reported, namely green for FHR increase and amber for FHR decrease. The length of a bar denotes the duration of the event to report. In the example of the window 324a, the length of the bar spans an interval of about 1 minute. Above the bar 326 is provided a control 330 that can be invoked to obtain more information on the event that the window denotes. The control also shows, in color and textually, the nature of the event, such as a late deceleration (L), a variable deceleration (V) or an acceleration (A) of the FHR amongst others. In addition, controls of different color intensities and containing letters other than the ones mentioned above may also be used to draw attention to events signalling potentially problematic situations. The control can be activated in different ways to show the additional information. One is to simply “click” with a pointing device on, or near, the control 330. In the case of a touch sensitive display 114, the physician can touch the area of the display 114 where the control 330 is shown to activate it. Yet another possibility is to activate the control 330 when the pointer of the pointing device hovers over, or near, the control 330. In this case there is no necessity to actually “click” on the control 330. As soon as the pointer is brought in proximity to the control 330, the activation occurs.

When the control 330 is activated, the window expands to take the shape shown in 324a. The entire area is then colored amber but remains translucent to keep the tracing 322 visible. In addition, a secondary window 332 pops up adjacent the primary window 324a, in which additional information appears. The additional information relates to the event of interest and can help the physician determine the relevance of the event denoted by the primary window 324a. Recall that the windows are actually generated by an algorithm which tracks the raw data, such as the FHR generated by the sensor 110. While most of the time the algorithm accurately identifies in the raw data the events of interest, it may occur that some events that are reported by a window are of little clinical relevance or even wrongly identified. A purpose of the secondary window 332 is to expose to the physician the raw data that has been used as a basis for the identification of the event, to allow the physician to validate the event. Specifically, the secondary window may convey the following type of information:
1. The type of event. In the examples shown the event can be a (late or variable) deceleration of the FHR or an acceleration of the FHR.
2. The duration of the event.
3. The magnitude of the rise or fall in the FHR within the event compared to the ambient baseline FHR value for instance a 16 Beats Per Minute (BPM) drop relative to the baseline BPM value that was observed before the event was triggered.
4. While not shown in the drawings, another element of information that can be used is the degree of confidence, reported by the algorithm in connection with the event recognition. This can be expressed as a percentage where the higher the percentage the higher the degree of confidence that the event is a real occurrence and not the result of an artifact in the raw data.

The window 324a is also associated with a second control, namely control 340, which allows editing the window 324a. In the example shown, the control is in the form of an “X” appearing near the bottom of the window 324a and when activated erases the window from the display 114. This option is used in instances where the window 324a is of little clinical relevance and may be removed to de-clutter the user interface 300. The control 340 is activated by “clicking” on it via a pointing device, such as a mouse, trackball or touch sensitive display surface.

Other editing options can be used as well. For example, the actuation of the control 340 can allow the physician to enter comments. This can be done by opening a text box in which comments can be typed. The comments are then stored and can be retained permanently as part of the records produced by the apparatus 100. Yet another editing option is to change the window size which corresponds to the duration of the event. That can occur when the algorithm has determined an event duration to be less than what it truly is and the physician can correct the duration by “dragging” the left or the right vertical line boundaries of the window to increase or decrease the duration. When the window 324a is edited in this fashion, the associated information that would appear in the secondary window 332 would also change. Specifically, the duration shown for the event will be corrected accordingly along with magnitude if the rise or fall in FHR within the event.

Yet another possible edit to the window 324a is to merge it with another adjacent window such that both windows are jointed into a single window.

As discussed earlier, the lower section 306, which primarily displays contractions rate information, is provided below the upper section 304, which is used primarily to display fetal heart rate information. Both sections are time synchronized. This allows correlating events reported in the upper section 304 to events occurring in the lower section 306. Specifically, window 324a has vertical bars 360 and 380 that delimit the window 324a horizontally. The bars 360 and 380 extend down up to the lower section 306 and intersect the tracing of the contraction rate. Therefore, the bars 360 and 380 show that the FHR deceleration event occurred somewhat before a contraction peak and dissipated shortly after the contraction ceased.

Below the lower section 306 are also provided a series of markers in the form of bars 400 that identify the occurrence of individual contractions. The bars 400 show the beginning and the end of each contraction and thus make the interpretation of the tracing easier. Specifically, one of the extremities of the bars 400 corresponds to the area of the tracing where a contraction begins and the opposite end of the bar 400 corresponds to the end of the contraction.

The use of the bars 400 is useful when the image is reduced in size, which can occur when the user interface is rendered on a small screen, such as a mobile computing device as a PDA. In such instances, the loss of resolution may be such as to make the tracing difficult to interpret and the bars 400 can be very useful to the user to spot where the contractions are.

Those skilled in the art should appreciate that in some embodiments of the invention, all or part of the functionality previously described herein with respect to the apparatus implementing a user interface for displaying labour related information may be implemented as pre-programmed hardware or firmware elements (e.g., application specific integrated circuits (ASICs), electrically erasable programmable read-only memories (EEPROMs), etc.), or other related components.

In other embodiments of the invention, all or part of the functionality previously described herein with respect to the apparatus for implementing a graphical user interface module for displaying labour related information may be implemented as software consisting of a series of instructions for execution by a computing unit. The series of instructions could be stored on a medium which is fixed, tangible and readable directly by the computing unit, (e.g., removable diskette, CD-ROM, ROM, PROM, EPROM or fixed disk), or the instructions could be stored remotely but transmittable to the computing unit via a modem or other interface device (e.g., a communications adapter) connected to a network over a transmission medium. The transmission medium may be either a tangible medium (e.g., optical or analog communications lines) or a medium implemented using wireless techniques (e.g., microwave, infrared or other transmission schemes).

The apparatus implementing a user interface for displaying labor related information may be configured as a computing unit 700 of the type depicted in FIG. 4, including a processing unit 702 and a memory 704 connected by a communication bus 708. The memory 704 includes data 710 and program instructions 706. The processing unit 702 is adapted to process the data 710 and the program instructions 706 in order to implement the functional blocks described in the specification and depicted in the drawings. In a non-limiting implementation, the program instructions 706 implement the functionality of processing unit 206 described above with reference to FIG. 2. The computing unit 700 may also comprise a number of interfaces 712a, 712b, 714, 716 for receiving or sending data elements to external devices. For example, interface 712b is used for receiving a data stream indicative of a fetal heart rate signal and interface 712a is for receiving a data stream indicative of a contraction signal. In addition, interface 714 is used for receiving a control signal from the user for controlling the information to be displayed on the graphical user interface implemented by the processing unit 702 when executing program instructions 706. Interface 716 is for releasing a signal causing a display unit to display the user interface generated by the processing unit 702 when executing program instructions 706. The computing unit shown in FIG. 4 may be part of any suitable computing device including, but not limited to, a desktop/laptop computing device or a portable digital assistant device (PDA).

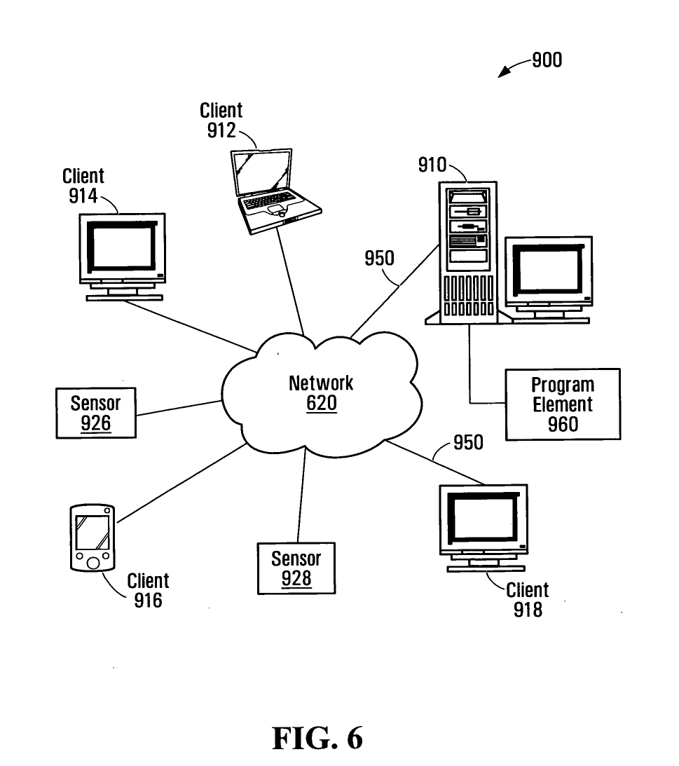

It will be appreciated that the system for implementing a user interface for displaying labour related information may also be of a distributed nature where the contraction signal and FHR signal are collected at one location by a uterine activity sensor and fetal heart rate sensor and transmitted over a network to a server unit implementing the graphical user interface. The server unit may then transmit a signal for causing a display unit to display the graphical user interface. The display unit may be located in the same location as the uterine activity sensor, in the same location as the server unit or in yet another location. FIG. 6 illustrates a network-based client-server system 900 for displaying uterine contraction information. The client-server system 900 includes a plurality of client systems 912, 914, 916, 918 connected to a server system 910 through network 920. The communication links 950 between the client systems 912, 914, 916, 918 and the server system 910 can be metallic conductors, optical fibers or wireless, without departing from the spirit of the invention. The network 920 may be any suitable network including but not limited to a global public network such as the Intranet, a private network and a wireless network. The server 910 may be adapted to process and issue signals to display multiple heart rate and contraction signals originating from multiple sensors 926, 928 concurrently using suitable methods known in the computer related arts.

The server system 910 includes a program element 960 for execution by a CPU. Program element 960 implements similar functionality as program instructions 706 (shown in FIG. 4) and includes the necessary networking functionality to allow the server system 910 to communicate with the client systems 912, 914, 916, 918 over network 620. In a non-limiting implementation, program element 960 includes a number of program element components, each program element components implementing a respective portion of the functionality of the user interface for displaying labor related information. FIG. 5 shows a non-limiting example of the architecture of program element 960 at the server system. As shown, the program element 960 includes four (4) program element components:
1. the first program element component 500 is executed on server system 910 and is for receiving signals conveying labor information;
2. the second program element component 502 is executed on server system 610 and is for sending messages to a client system, say client system 914, for causing client system 914 to display the user interface as described in connection with FIG. 3;
3. the third program element component 504 is executed on server system 910 and is for sending messages to client system 914 for causing client system 914 to display controls 330 and 340 (shown in FIG. 3);
4. the fourth program element component 506 is executed on server system 910 and is for receiving a message from client system 914 indicative of an actuation of anyone of the controls 330 and 340 (shown in FIG. 3).

Those skilled in the art should further appreciate that the program instructions may be written in a number of programming languages for use with many computer architectures or operating systems. For example, some embodiments may be implemented in a procedural programming language (e.g., “C”) or an object oriented programming language (e.g., “C++” or “JAVA”).

Although various embodiments have been illustrated, this was for the purpose of describing, but not limiting, the invention. Various modifications will become apparent to those skilled in the art and are within the scope of this invention, which is defined more particularly by the attached claims.

1) A computer readable storage medium holding a program element for execution by a computer to implement a user interface conveying fetal condition information to a user, the user interface comprising a first section showing one or more vital signs of a fetus, said section including:
a) at least one tracing associated with a vital sign;
b) a marker to visually highlight an area of the tracing corresponding to an event of interest;
c) a control component that can be activated by the user to deliver additional information about the event of interest.

2) A computer readable storage medium as defined in claim 1, wherein the vital sign is the FHR.

3) A computer readable storage medium as defined in claim 2, wherein the marker has a dimension corresponding to a duration of the event of interest.

4) A computer readable storage medium as defined in claim 3, wherein the event of interest is an FHR deceleration.

5) A computer readable storage medium as defined in claim 3, wherein the event of interest is an FHR acceleration.

6) A computer readable storage medium as defined in claim 1, wherein the additional information is delivered via a text box appearing on the display.

7) A computer readable storage medium as defined in claim 6, wherein the text box displays the duration of the event of interest.

8) A computer readable storage medium as defined in claim 6, wherein the text box displays an average FHR variation over the duration of the event of interest.

9) A computer readable storage medium as defined in claim 6, wherein the text box displays a confidence factor.

10) A computer readable storage medium as defined in claim 1, wherein the control component is a first control component, the user interface further including a second control component which can be activated to allow a user to edit the marker.

11) A computer readable storage medium as defined in claim 10, wherein the second control component allows the user to erase the marker.

12) A computer readable storage medium as defined in claim 1, wherein said section is a first section, said user interface further including a second section showing a contraction information.

13) A computer readable storage medium as defined in claim 12, wherein the first section and the second sections are time synchronized.

14) A computer readable storage medium as defined in claim 1, wherein the user interface is rendered on a display of a mobile computing device.

15) A computer readable storage medium as defined in claim 14, wherein the mobile computing device includes a PDA.

16) An apparatus for conveying fetal condition information during labour, said apparatus comprising:
a) an first input for receiving a contraction signal;
b) a second input for receiving a fetal heart rate signal;
c) a processing unit in communication with said first and second inputs, said processing unit implementing a user interface conveying fetal condition information to a user, the user interface comprising a section showing one or more vital signs of a fetus, said section including:
i) at least one tracing associated with the fetal heart rate signal;
ii) a marker to visually highlight an area of the tracing corresponding to an event of interest;
iii) a control component that can be activated by the user to deliver additional information about the event of interest;
d) an output for releasing control information for causing the user interface to be displayed on a display unit.

17) An apparatus as defined in claim 16, said apparatus further comprising a third input for receiving data from the user for activating the control component on the user interface.
